(19) United States
(12) Patent Application Publication
(10) Pub. No.: US 2025/0195724 A1
(43) Pub. Date: Jun. 19, 2025

(54) HYDROGEL COMPOSITES AND METHODS OF MAKING SAME

(71) Applicants: Emily Elizabeth EARL, Victoria (CA); Mina HOORFAR, Victoria (CA)

(72) Inventor: Emily Elizabeth EARL, Victoria (CA)

(21) Appl. No.: 18/977,731

(22) Filed: Dec. 11, 2024

Related U.S. Application Data
(60) Provisional application No. 63/609,476, filed on Dec. 13, 2023.

(51) Int. Cl. A61L 27/52; A61L 27/04; A61L 27/14; A61L 27/44; A61L 27/56

(52) U.S. Cl. CPC A61L 27/52; A61L 27/04; A61L 27/14; A61L 27/446; A61L 27/56; A61L 2400/06

(57) ABSTRACT

A hydrogel composite is disclosed. The hydrogel composite is biocompatible and swellable. The hydrogel composite may have a swollen density greater than gastric fluid, for example, greater than about 1.05 g/mL. The high-density hydrogel composite may be used to relocate contents of the gastric acid pocket to the bottom of the stomach so as to allow for better distribution of gastric acid. The hydrogel composite comprises a polymer and a high-density material incorporated with the polymer. The polymer may be modified by a cross-linking agent. Methods of making the hydrogel composite are also disclosed.

### CROSS-REFERENCE TO RELATED APPLICATION

This application claims the benefit under 35 U.S.C. § 119 of U.S. application No. 63/609,476 filed 13 Dec. 2023 and entitled HYDROGEL COMPOSITES AND METHODS OF MAKING SAME which is hereby incorporated herein by reference for all purposes.

### FIELD OF THE INVENTION

The invention pertains to hydrogel composites, in particular, those for use in the treatment of heartburn.

### BACKGROUND

Treatments for heartburn, or those who suffer from gastroesophageal reflux disease (GERD), are known in the art. Heartburn is a symptom that is caused by gastric fluid entering the esophagus and damaging the esophageal lining. Conventional treatments include over-the-counter antacids which act to neutralize the acidity of the stomach contents, thereby raising the pH of the refluxate closer to the native pH of the lower esophagus. The long-term use of pH-balancing antacids is not recommended due to unfavourable side effects. Another conventional treatment for heartburn includes a drug which acts to inhibit the body's ability to produce hydrochloric acid. While these drugs are designed to alleviate the pain caused by acid reflux, they cause the natural pH of the stomach to be altered for time periods ranging from a few months to multiple decades. Although it was believed that one of the causes of frequent heartburn was the result of an individual's heightened ability to produce gastric acid, this is not the case in the majority of individuals. Often, the cause of frequent heartburn is the result of a dysfunction of the lower esophageal sphincter, which causes the sphincter to relax at abnormal intervals. For individuals who experience esophageal malfunction and do not over-produce hydrochloric acid, the exact implications of the gastric acid reduction caused by acid inhibitors are unknown. The present invention is directed to an improved treatment for heartburn.

### SUMMARY

The invention provides a biocompatible, swellable, hydrogel composite. The hydrogel composite may have a swollen density greater than gastric fluid, for example, greater than about 1.05 g/mL. The hydrogel composite comprises a polymer and a high-density material incorporated with the polymer. The polymer may be modified by a cross-linking agent.

In some example embodiments, the polymer comprises an ionic polymer having a polar functional group. The polar functional group may be one or more of a hydroxyl group (—OH), a carboxyl group (—COOH), and an amino group (—NH2). In some embodiments, the polymer comprises a cationic polymer. In some example embodiments, the cationic polymer is one or more of chitosan, poly(ethyleneimine), poly(amino-co-ester)s, poly(amidoamine)s and poly-L-(lysine).

In some embodiments, the cross-linking agent comprises a dialdehyde compound, such as for example glutaraldehyde and/or glyoxal.

In some embodiments, the high-density material comprises density greater than about 3 g/mL, or greater than about 4 g/mL. In some example embodiments, the high-density material comprises a metal oxide. Non-limiting examples of molecules that may be selected as the high-density material include one or more of titanium dioxide, zirconium dioxide, barium sulfate, zinc oxide, and iron powder.

Aspects of the invention pertain to methods of making a hydrogel composite. In some embodiments, the method involves synthesizing the hydrogel composite using a gas-blowing method. In some embodiments, the method involves synthesizing the hydrogel composite using a freeze-drying method. In some embodiments, the method comprises mechanically compressing the hydrogel composite. The mechanical compression of the hydrogel composite may comprise incorporating the hydrogel composite into a confined space. The confined space comprises a volume less than a volume of the hydrogel composite. In some embodiments, the confined space comprises a pill, tablet, capsule and the like.

One application of the hydrogel composite pertains to the treatment of heartburn, for example, individuals who suffer from gastroesophageal reflux disease (GERD).

Further aspects of the invention and features of specific embodiments of the invention are described below.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic diagram illustrating a hydrogel composite according to an example embodiment of the invention.

FIG. 2A is a photograph showing the hydrogel composite made using a gas-blown method after the step of cross-linking the polymer.

FIG. 2B is a photograph showing the hydrogel composite made using a gas-blown method after the step of submerging the composite in an ethanol bath.

FIG. 2C is a photograph showing the hydrogel composite made using a gas-blown method after the step of drying the composite in an oven.

FIG. 3 is a graph showing the swelling ratio (%) over time (minutes) for each of Samples 1 to 6 in pH 1.57 hydrochloric acid solution.

FIG. 4 is a graph showing the swelling ratio (%) over time (minutes) for each of Samples 7 to 10 in pH 1.57 hydrochloric acid solution.

FIG. 5 shows image analysis of a micro-CT scan of a hydrogel composite made using a gas-blown method with a) an image during mask selection, b) an image converted to black and white, and c) a resulting image after applying a threshold.

FIG. 6 is a plot showing the swelling ratios (%) of first-generation hydrogel composites made using a freeze-drying method for each of Samples 1 to 6 in pH 1.57 hydrochloric acid solution. Solid bars indicate that the hydrogel composites possessed a density greater than the density of gastric fluid.

FIG. 7 shows image analysis of a micro-CT scan of a hydrogel composite made using a freeze-drying method with a) an unaltered image from a micro-CT scan, b) an image converted to black and white, and c) a resulting image after applying a threshold.

FIG. 8 are SEM images of first-generation hydrogel composites made using a freeze-drying method without zirconia with a) 200× magnification, b) 1000× magnification, and with zirconia, c) 200× magnification, and d) 1000× magnification.

FIG. 9 shows FTIR spectra of first-generation hydrogel composites made using a freeze-drying method with 15% w/v zirconia and without zirconia.

FIG. 10 show images of cytotoxicity results, with a) first generation hydrogel composites made using a freeze-drying method without zirconia, and b) first generation hydrogel composites made using a freeze-drying method with zirconia.

FIG. 11 is a graph showing the expectant swelling ratios (%) of second-generation hydrogel composites made using a freeze-drying method for each of Samples 1-3 in pH 1.57 hydrochloric acid solution.

FIG. 12 is a plot showing the swelling ratios (%) over time (minutes) to illustrate an effect of solution pH on swelling ability of optimized hydrogel.

FIG. 13A are schematic diagrams showing the dimensions of compressed versus uncompressed hydrogel composites.

FIG. 13B is a plot showing the swelling ratios (%) for compressed versus uncompressed hydrogel composites, illustrating their swelling abilities in pH 1.57 hydrochloric acid solution.

FIG. 14 shows image analysis of a micro-CT scan of a second-generation hydrogel composite made using a freeze-drying method with a) an unaltered image from a micro-CT scan, b) an image converted to black and white, and c) a resulting image after applying a threshold.

FIG. 15 are SEM images of second-generation hydrogel composites made using a freeze-drying method without zirconia with a) 1000× magnification, b) 5000× magnification, and with zirconia, c) 1000× magnification, and d) 5000× magnification.

FIG. 16 shows FTIR spectra of pure zirconia and second-generation hydrogel composites made using a freeze-drying method with 4.5% w/v zirconia and without zirconia.

FIG. 17 show X-Ray diffraction (XRD) data of pure zirconia and second-generation hydrogel composites made using a freeze-drying method with 4.5% w/v zirconia and without zirconia.

FIG. 18A show cytotoxicity results, with live or dead staining of 0% and 4.5% w/v zirconia hydrogel composites at day 1 and day 5.

FIG. 18B is a plot of cell viability (%) at day 1 and day 5 for each of 0% and 4.5% w/v zirconia hydrogel composites.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

### Definitions

“Swollen density” is the density of the hydrogel composite after submersion in test medium. The swollen density is calculated by the following formulae:

$${d}_{s} = \frac{{M}_{s}}{{V}_{i}}$$

where Mₛ represents the swollen mass of the hydrogel composite (or mass of the hydrogel composite after submersion in test medium), and Vᵢ represents the volume of the hydrogel composite. In one non-limiting example, the swollen density of the hydrogel composite is determined after the composite is submerged in hydrochloric acid with a pH of about 1.2 to simulate gastric fluid.

“Swelling ratio” (%) is the difference between the initial weight of the dry hydrogel composite and the weight of fully swollen hydrogel composite divided by the initial weight of the dry hydrogel composite, calculated by the following formulae:

$$Swelling Ratio ( \% ) = \frac{{M}_{s} - {M}_{d}}{{M}_{d}} * 100$$

where Mₛ represents the swollen mass of the hydrogel composite, and Ma represents the initial dry mass of the hydrogel.

“Swelling capacity” or “swelling ability” is the capacity or ability of the hydrogel composite to absorb test media, or swell in the test media, as determined by the swelling ratio.

“Equilibrium swelling time” refers to the point in time at which a hydrogel composites reached a constant swelling ratio.

### Example Embodiment of Hydrogel Composite

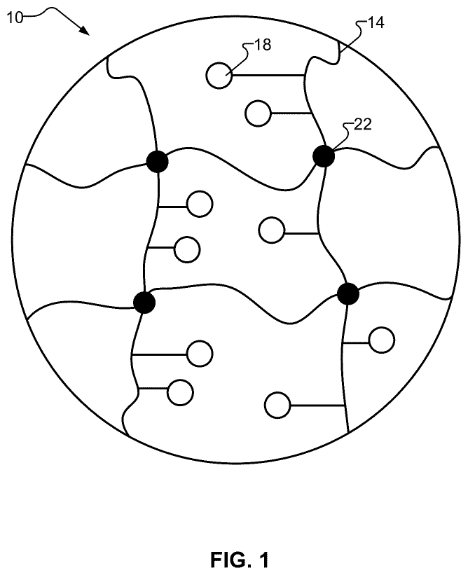

Referring to FIG. 1, in one embodiment, the invention is a hydrogel composite 10. A specific application of the hydrogel composite 10 is in the treatment of heartburn, such as those who suffer from gastroesophageal reflux disease (GERD). The hydrogel composite 10 may be used to relocate contents of the gastric acid pocket to the bottom of the stomach so as to allow for better distribution of gastric acid. This may result in a higher pH of the gastric fluid near the esophagus, advantageously reducing the likelihood of heartburn.

The hydrogel composite 10 comprises a polymer 14 and a high-density material 18. A polymer is a molecule composed of at least two repeating structural units which may be joined by chemical bonds. The polymer 14 may be a natural polymer and/or a synthetic polymer. In some embodiments, the polymer 14 is an ionic polymer (i.e., polymers which contain ions in the polymer chains). In some embodiments, the polymer 14 is a hydrophilic polymer (i.e., polymers which contain polar functional groups in the polymer chains). In some example embodiments, the polymer 14 contains one or more of hydroxyl groups (—OH), carboxyl groups (—COOH) and amino groups (NH₂). The polymer 14 may contain functional groups which are anionic (i.e., contains negative ions) and/or cationic (i.e., contains positive ions). In one example embodiment, the polymer 14 is a cationic polymer (i.e., a polymer having positively charged ions). The positive ions contained in the cationic polymer are protonated and become positively charged in a low pH environment. The inventor believes that cationic polymers facilitate swelling of the molecule in a low pH environment. Non-limiting examples of cationic polymers that may be used as the polymer 14 include chitosan, poly(ethyleneimine), poly(amino-co-ester)s, poly(amidoamine)s and poly-L-(lysine).

A molecule that may be selected as the polymer 14 preferably has one or more of the following characteristics:
biodegradable; and/or
biocompatible; and/or
digestible by bodily enzymes; and/or
good mucoadhesive abilities; and/or
cationic; and/or
naturally-occurring, etc.

In some example embodiments, the polymer 14 comprises chitosan. Chitosan is a linear polysaccharide composed of randomly distributed β-(1→4)-linked d-glucosamine (deacetylated unit) and N-acetyl-d-glucosamine (acetylated unit). A segment of the structure of chitosan is illustrated below:

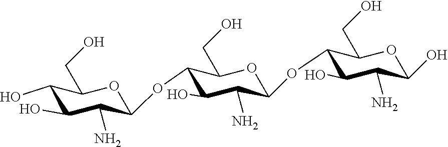

In some embodiments, the polymer 14 is modified by a cross-linking agent 22 to produce a cross-linked polymer. A cross-linked polymer may be a polymer with one or more cross-link bonds being between the monomeric units of the polymer. The cross-linking agent 22 may be a chemical agent and/or a physical agent. In some embodiments, the cross-linking agent 22 is a chemical agent. The chemical agent may be a chemical compound such as a dialdehyde. However, any cross-linking agent that is suitable for cross-linking the selected polymer with the desired mechanical and/or chemical properties may be used. The suitable cross-linking agent may for example be selected based on the molecule(s) selected for use as the polymer 14. In some example embodiments in which chitosan is selected as the polymer 14, the cross-linking agent 22 comprises glutaraldehyde and/or glyoxal. Glutaraldehyde and/or glyoxal may for example be used to chemically modify the chitosan polymer by connecting the monomers through their amine groups as illustrated below:

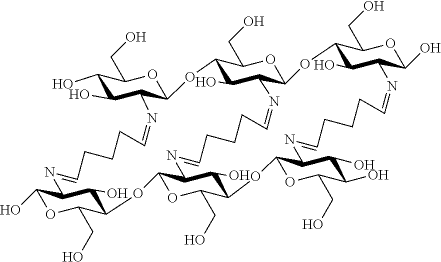

In some embodiments, the concentration of the polymer 14 is in the range of from about 0.5% w/v to about 5% w/v, or in some embodiments, in the range of from about 0.5% w/v to about 3% w/v, or in some embodiments, in the range of from about 0.5% w/v to about 1.5% w/v. In some embodiments, the concentration of the polymer 14 is about 1% w/v. In some embodiments, such polymer 14 comprises chitosan. The concentration of the polymer 14 may be adjusted depending on the type and/or nature of the polymer selected. In some embodiments, the polymer 14 comprises a concentration of up to about 70% w/v.

In some embodiments, the weight ratio of the cross-linking agent 22 to the polymer 14 is not greater than about 2% w/w, and in some embodiments, in the range of from about 0.30% to about 0.60% w/w, and in some embodiments, about 0.5% w/w.

The high-density material 18 may be incorporated with the polymer 14. In some embodiments, the high-density material 18 is bonded to the polymer 14. The high-density material 18 is a material which combines with the polymer 14 to achieve a hydrogel composite 10 with a density that is greater than the density of gastric fluid, such that the hydrogel composite 10, when it reaches the stomach, sinks to the bottom of the stomach. The hydrogel composite 10 preferably comprises a density sufficient to withstand the movement of peristalsis in the stomach, thereby preventing the composite 10 from being ejected from the stomach during peristalsis. In some embodiments, the swollen density of the hydrogel composite 10 is at least about 1.05 g/mL, or in some embodiments, at least about 1.5 g/mL, or in some embodiments, in the range of from about 1.5 g/mL to about 5 g/mL, or in some embodiments, in the range of from about 2.5 g/mL to about 3 g/mL.

In some embodiments, the high-density material 18 has a density of at least about 2 g/mL, or in some embodiments, at least about 3 g/mL, or in some embodiments, at least about 4 g/mL. In some embodiments, the high-density material 18 has a density in the range of from about 1 g/mL to about 10 g/mL, or in some embodiments, in the range of from about 2 g/mL to about 8 g/mL, or in some embodiments, in the range of from about 3 g/mL to about 6 g/mL.

The high-density material 18 comprises one or more materials that are biocompatible (i.e., the material is compatible with biological tissues) and/or bioinert (i.e., the material does not elicit a response when in contact with biological tissues). In some embodiments, the high-density material 18 comprises a ceramic material. In some embodiments, the high-density material 18 comprises a metal oxide. Non-limiting examples of compounds that may be selected as the high-density material 18 include titanium dioxide, zirconium dioxide, barium sulfate, zinc oxide, iron powder, etc. The high-density material 18 may comprise a plurality of compounds. In some example embodiments in which chitosan is used as the polymer 14, titanium dioxide and/or zirconium dioxide is selected for use as the high-density material 18. The inventor believes that weak hydrogen bonds may be formed between titanium dioxide or zirconium dioxide molecules and the —OH and —NH2 groups of the chitosan chains when titanium dioxide or zirconium dioxide is incorporated with chitosan. Titanium dioxide has a density of about 4.23 g/mL. Zirconium dioxide has a density of about 5.68 g/mL.

In some embodiments, the concentration of the high-density material 18 in the hydrogel composite 10 is greater than about 2% w/v, and in some embodiments, greater than about 3% w/v, and in some embodiments, greater than about 4% w/v. In some embodiments, the concentration of the high-density material 18 in the hydrogel composite 10 is in the range from about 3% w/v to about 30% w/v, and in some embodiments, in the range from about 3% w/v to about 20% w/v, and in some embodiments, in the range from about 3.5% w/v to about 10% w/v, and in some embodiments, in the range from about 3.5% to about 5.5%.

In some embodiments, the hydrogel composite 10 is in a deformed or compressed state. The hydrogel composite 10 for example can be mechanically deformed or compressed such as by flattening, from a natural state thereof. The natural state may be defined by an original volume. In some embodiments, the hydrogel composite 10 is compressed to about 5% to about 50% of the original volume, and in some embodiments, about 5% to about 20%, and in some embodiments, about 10% to about 20%. In some embodiments, the hydrogel composite 10 is deformed or compressed to a size of a capsule which is suitable for oral administration. In some embodiments, the hydrogel composite 10 has a volume in the range of from about 0.1 mL to about 5 mL, and in some embodiments, between about 0.2 mL to about 3 mL, and in some embodiments, between about 0.5 mL to about 2 mL, and in some embodiments, between about 0.68 mL to about 1.37 mL.

A desirable hydrogel composite 10 for use to relocate contents of the gastric acid pocket to the bottom of the stomach has one or more of the following properties in low pH environments such as with a pH of about 1.2:
highly swellable, with a swelling ratio of greater than at least 1000% in some embodiments, and in some embodiments, greater than 1500%, and in some embodiments, greater than about 2000%, and in some embodiments, greater than 2500%; and/or
a low equilibrium swelling time, for example of less than one hour, and in some embodiments less than 45 minutes, and in some embodiments less than about 30 minutes, and in some embodiments less than about 15 minutes, and in some embodiments less than about 5 minutes, and in some embodiments about 1 minute with 25 g/g gastric fluid; and/or
a swollen density of greater than 1 g/mL, and in some embodiments, greater than about 1.05 g/mL; and/or
high porosity, with an average porosity of greater than about 70% in some embodiments, and greater than about 80% in some embodiments, and in some embodiments, greater than about 85%, and in some embodiments, greater than about 90%; and/or
large pore sizes, with an average pore size of greater than about 100 μm, and in some embodiments, in the range of from about 50 μm to about 5000 μm, and in some embodiments, from about 50 μm to about 2500 μm, and in some embodiments, from about 100 μm to about 1000 μm, and in some embodiments, from about 100 μm to about 500 μm, and in some embodiments, about 100 μm to about 300; and/or
mass of at least 2 grams, and in some embodiments, at least 3 grams, and in some embodiments, at least about 4 grams, and in some embodiments, in the range of from about 2 grams to about 10 grams, and in some embodiments, in the range of from about 2 grams to about 5 grams; and/or
biocompatible which may be demonstrated in cell cytotoxicity assays.

The properties of the hydrogel composite 10 (e.g., cell cytotoxic behavior, swollen density, biocompatibility, structural strength, elasticity, swelling ratio or capability, pore sizes, and/or porosity, etc.) may be optimized by adjusting one or more of:
nature of the polymer 14, cross-linking agent 22, high-density material 18; and/or
the weight ratio of the cross-linking agent 22 to the polymer 14; and/or
the weight ratio of the polymer 14 and/or cross-linking agent 22 to the high-density material 18; and/or
the concentration(s) of each of the polymer 14, cross-linking agent 22, high-density material 18; and/or
method of making the hydrogel composites 10; and/or
the temperature at which the hydrogel composites 10 are frozen prior to lyophilization (if applicable); and/or
addition of other compounds to the hydrogel composite, for example, swelling compounds including synthetic superabsorbent polymers including poly (acrylic acid), poly (methacrylic acid), poly (ethylene glycol), polyacrylamide, and sodium polyacrylate, and natural superabsorbent polymers including alginate, starch, guar gum, xanthan gum, cellulose, and carrageenan, etc.
etc.

### Example Methods of Making Hydrogel Composite

Aspects of the invention involves making a hydrogel composite. The hydrogel composite comprises a polymer and a high-density material. In some embodiments, the polymer is modified by a cross-linking agent to produce a cross-linked polymer.

In some embodiments, the method of making a hydrogel composite involves a gas blowing method. In such embodiments, the method comprises preparing one or more polymeric solutions. In some example embodiments, the one or more polymeric solutions comprises a first polymeric solution comprising chitosan. The chitosan may for example be dissolved in a solvent such as an acid solution (e.g., acetic acid). In some example embodiments, the one or more polymeric solutions comprises a second polymeric solution comprising poly(vinyl alcohol) (PVA). In such embodiments, the second polymeric solution is added to improve hydrogel structure and swelling ability. Without being bound to any theory, PVA may create a semi-interpenetrating polymer network so as to increase the structural integrity of the hydrogel. In addition, the hydrophilic nature of PVA may advantageously increase the swelling of the hydrogel. In such embodiments, the PVA is dissolved in a solvent, such as water. The method comprises combining a high-density material with the polymeric solutions. The pH of the solution may be adjusted. In some embodiments, the pH of the solution is lowered to about 5.0 to prepare the conditions for the cross-linking reaction between the cross-linking agent and the one or more polymers. The pH may be adjusted by the addition of an acid, such as acetic acid. A solution comprising a cross-linking agent may be prepared, and then added to the solution comprising the high-density material with the one or more polymers to produce the hydrogel composite. In some embodiments, a foaming agent is added to the hydrogel composite to generate pores therein. In some example embodiments, the foaming agent comprises a base. The base may for example be sodium bicarbonate. The hydrogel composite may be submerged in alcohol, such as an ethanol bath. The alcohol-submerged hydrogel composite may then be dried. The drying of the hydrogel composite may be performed in a dryer such as an oven at a temperature in the range of from about 40° C. to about 80° C., and in some embodiments, about 50° C. to 60° C.

In some embodiments, the method of making a hydrogel composite involves a freeze-drying method. In such embodiments, the method comprises preparing one or more polymeric solutions. In some example embodiments, the one or more polymeric solutions comprises a first polymeric solution comprising chitosan. The chitosan may for example be dissolved in a solvent such as an acid solution (e.g., acetic acid). A solution comprising a cross-linking agent may be prepared. The one or more polymeric solutions, the solution comprising the cross-linking agent, and the high-density material may be combined to form a mixture. The mixture may be left at room temperature for a time interval (e.g., about 30 minutes) sufficient to allow the polymer to cross-link. The mixture may then be placed at a freezing temperature (e.g., at a temperature of about −80° C.) to allow the mixture to freeze. The frozen mixture may then be lyophilized (freeze-dried) to form the hydrogel composite, for example in a freeze dryer for a time interval (e.g., about 12 to 24 hours).

In some embodiments, the method of making the hydrogel composite comprises combining the gas blowing method and the freeze-drying method. In such embodiments, certain steps of the two methods may be combined to synthesize the hydrogel composite. In some embodiments, a foaming agent is introduced into the polymeric mixture to generate additional pores therein before the mixture is placed at a freezing temperature to form a frozen mixture. The frozen mixture may then be lyophilized (freeze-dried) to form the hydrogel composite.

In some embodiments, the method further comprises mechanically compressing the hydrogel composite. The inventor has found that mechanically compressing the hydrogel composite increases the swelling capacity of the hydrogel composites. The hydrogel composite may be mechanically compressed by incorporating the hydrogel composite into a confined space. The confined space comprises a volume smaller than a volume of the hydrogel composite. The confined space may for example be in the form of a capsule, tablet, pill, and the like. In some embodiments, the confined space has a volume in the range of from about 0.1 mL to about 5 mL, and in some embodiments, between about 0.2 mL to about 3 mL, and in some embodiments, between about 0.5 mL to about 2 mL, and in some embodiments, between about 0.68 mL to about 1.37 mL.

In some embodiments, the method further comprises adding one or more compounds in the synthesis of the hydrogel composite. Such one or more compounds include for example swelling compounds, which may be added to improve the swelling capacity of the hydrogel composite. The one or more compounds may be added at any suitable step(s) in the synthesis of the composite. For example, the one or more compounds may be added at the time of combining the high-density material with the one or more polymeric solution.

Aspects of the invention pertain to use of the hydrogel composite in the treatment of heartburn, for example, in the treatment of individuals who suffer from gastroesophageal reflux disease (GERD). The hydrogel composite may be formulated for oral administration. The hydrogel composite may for example be formulated in the form of a tablet, pill, capsule, and the like. Alternatively, the hydrogel composite may be protected using waterproof sprays or coatings for formulating in the form of an oral suspension, and the like.

### Examples

### Example 1. Gas Blowing Method

### Materials

Chitosan (medium molecular weight, 75-85% deacetylated), titanium(iv) oxide nanopowder (21 nm), glyoxal (40% in water), poly(vinyl alcohol) (99+% hydrolyzed), and sodium bicarbonate were purchased from Sigma Aldrich. Hydrochloric acid (2.0N) and glacial acetic acid were purchased from VWR. All aqueous solutions were prepared with reverse osmosis (RO) purified water and all other chemicals used were of analytical grade.

### Hydrogel Synthesis

A hydrogel composite 10 of the type illustrated in FIG. 1 was synthesized using a gas-blown method. The polymer 14 comprises chitosan and poly(vinyl alcohol) (PVA). The high-density material 18 comprises titanium dioxide. The cross-linking agent 22 comprises glyoxal.

A 2% w/w solution of chitosan was prepared by dissolving chitosan in 1% v/v acetic acid at 60 degrees Celsius under magnetic stirring for a minimum of 5 hours. Additionally, a 10% w/w solution of poly(vinyl alcohol) (PVA) was prepared by dissolving PVA in RO water at 95 degrees Celsius under magnetic stirring for 30 minutes. A solution of 10% w/w glyoxal was used as the crosslinker, titanium dioxide nanoparticles were used as the high-density material, and sodium bicarbonate was used as the foaming agent. The chitosan, PVA, and titanium dioxide particles were combined under magnetic stirring for a minimum of 30 minutes to ensure that the solution was well mixed. The pH of the solution was then lowered to 5.0 by the dropwise addition of 0.1 M acetic acid to ensure the reaction between glyoxal and chitosan would occur. The glyoxal was then added to the mixture which was shaken vigorously and after 30 seconds sodium bicarbonate was added to induce bubble generation. The hydrogel was left to rest for 30 minutes and then placed into an ethanol bath for at least 5 hours before being dried in an oven overnight at 55 degrees Celsius.

The chitosan, PVA, glyoxal, and titanium dioxide were combined in varying amounts, as listed in Table 1 below. Titanium dioxide was only added to Samples 5 and 6 to compare how the titanium dioxide affected the swelling ability of the hydrogels.

TABLE 1
Gas-blown hydrogel formulations: comparison of polymer percentage,
cross-linker percentage, and percentage of titanium dioxide

| | | | | Titanium | Sodium |
|---|---|---|---|---|---|
| | Chitosan | PVA | Glyoxal | Dioxide | Bicarbonate |
| | (mg) | (mg) | (mg) | (mg) | (mg) |
| Sample 1 | 400 | 160 | 400 | 0 | 80 |
| Sample 2 | 400 | 160 | 160 | 0 | 80 |
| Sample 3 | 160 | 400 | 400 | 0 | 80 |
| Sample 4 | 160 | 400 | 160 | 0 | 80 |
| Sample 5 | 160 | 400 | 400 | 20 | 80 |
| Sample 6 | 160 | 400 | 400 | 50 | 80 |

After the first experiment, another experiment was prepared to try and increase the swelling ability of the hydrogels further. In the second experiment, the PVA was removed from the hydrogels, a foam stabilizer, Pluronic F127, was added into one of the mixtures, and the amount of glyoxal was varied. In this experiment, the titanium dioxide was excluded from the study in an attempt to identify the ideal crosslinker percentage as well as to determine the effects of the foam stabilizer. The individual sample volumes were also increased to increase the ease of handling. The different sample sets are shown in Table 2.

TABLE 2
Gas-blown hydrogel formulations: comparison of cross-
linker percentage and effects of foam stabilizer

| | Chitosan | Glyoxal | Pluronic F 127 | Sodium Bicarbonate |
|---|---|---|---|---|
| | (ml) | (ml) | (% w/w) | (mg) |
| Sample 7 | 3 | 0.12 | 0 | 50 |
| Sample 8 | 3 | 0.12 | 0.5 | 50 |
| Sample 9 | 3 | 0.9 | 0 | 50 |
| Sample 10 | 3 | 0.6 | 0 | 50 |

### Results

### Hydrogel Synthesis

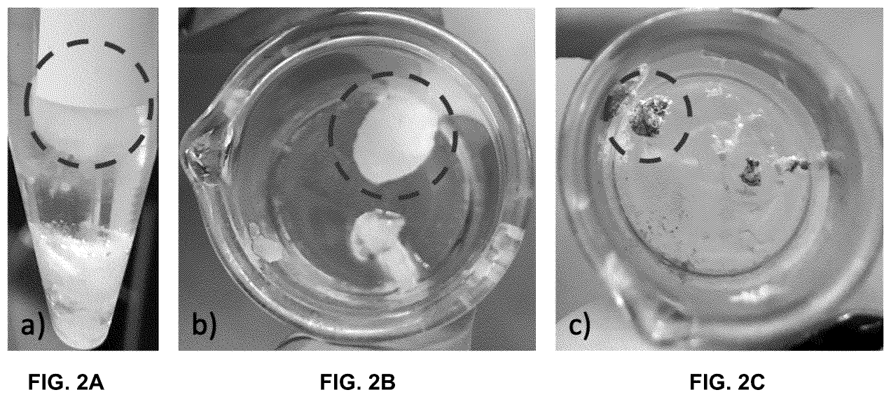

Immediately after crosslinking, the hydrogels congealed within the solutions. The hydrogels that were made with a high amount of glyoxal formed a singular solid mass that floated at the top of the solution (FIG. 2A), whereas the hydrogels that were made with a low amount of glyoxal turned the solution into a slurry of semi-solid material. After drying in the oven overnight, the samples shrunk significantly and changed from a clear, gelatinous substance to a brown, brittle material (FIG. 2C). The reduction in size occurs due to the removal of liquid from the hydrogel and the change in colour was a result of the heat treatment of the chitosan. When the hydrogels were placed into hydrochloric acid to swell, they did not return to their original form that was expressed after the crosslinking process; the hydrogels remained small and brittle for the full 30-minute testing period and no visible changes in size were observed for any of the samples.

### Swelling Studies

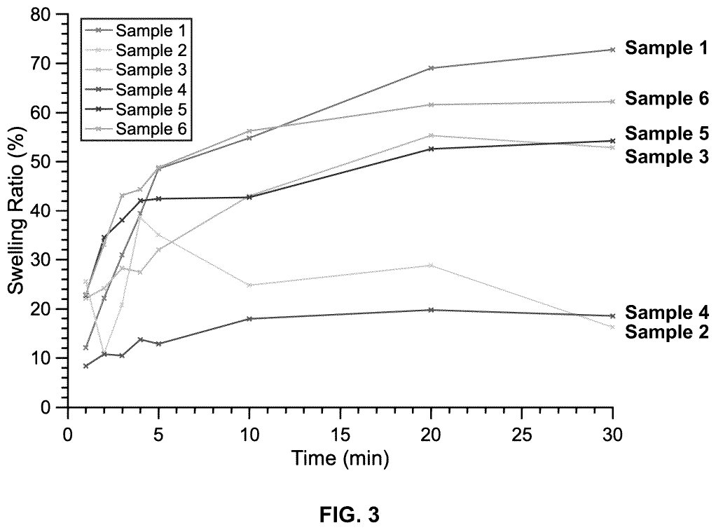

The results from the initial swelling tests done with the gas-blown hydrogels in pH 1.57 hydrochloric acid solution are shown in FIG. 3. Both Sample 2 and Sample 4 performed very poorly compared to the other hydrogels, yielding a final swelling ratio of just under 20% after the 30-minute period. Sample 2 and Sample 4 were the only samples that had the lower amount of glyoxal used during hydrogel formation, indicating that a higher amount of crosslinker was ideal for maximized swelling. Usually, an increased amount of glyoxal would lead to decreased swelling ability due to a more tightly bound hydrogel network [170]. While the observed trend went against what was to be expected, it was assumed to occur as a result of the drying process. With oven-dried hydrogels, the inventor believes that it is common to observe a more wrinkled and collapsed hydrogel structure due to the volume change in the hydrogel. The increased strength of the polymer network in the hydrogels made with a larger amount of glyoxal may have prevented the generated pores within the hydrogel from collapsing completely during the drying process; however, in the samples synthesized with a lower amount of glyoxal, the hydrogel network may not have been strong enough to sustain the porous structure while drying, resulting in a complete collapse of the pores and limited swelling ability. Out of the six samples, Sample 1 performed the best, yielding a swelling ratio just above 70%. Sample 1 had the highest amount of both chitosan and glyoxal, suggesting that an increase in chitosan also increased the swelling ability of the hydrogels. The formulations of Samples 3, 5, and 6 were identical aside from the mass of titanium dioxide added which increased with sample number. As Sample 6 showed the highest swelling ability of the three, this indicated that the titanium dioxide may also help in maintaining the porous structure of the hydrogel. Additionally, none of the titanium dioxide was observed to separate from the hydrogel during the swelling process, indicating that the titanium dioxide was successfully integrated with the chitosan hydrogel.

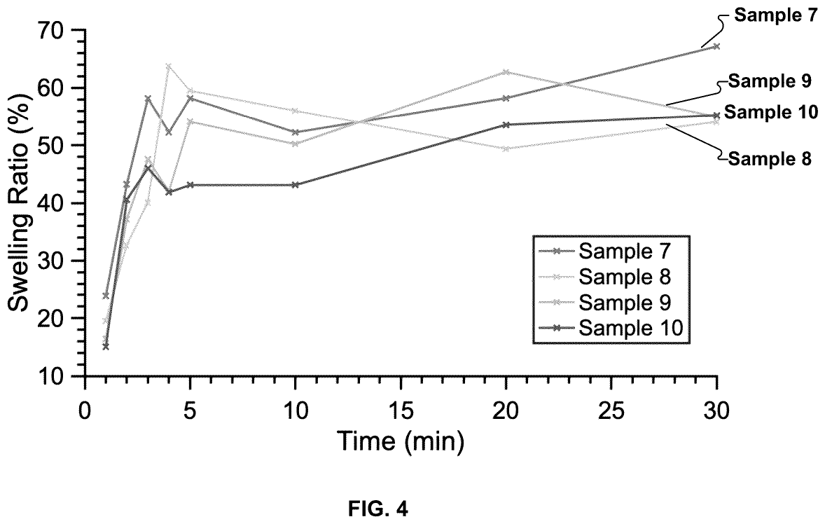

The results from the second experiment in pH 1.57 hydrochloric acid are shown in FIG. 4. The results follow the same trend as the first experiment: an increase in the concentration of glyoxal in the hydrogel resulted in increased swelling. Additionally, while adding foam stabilizer appeared to increase the swelling ratio at the beginning of the test, it did not seem to significantly affect the swelling of the hydrogel overall. This suggests that the foam stabilizer may not be able to combat the pore destruction from the drying process. Based on the swelling patterns visible from both tests one and two, it seems as if the hydrogels may have swollen further given additional time in the media; however, yielding a swelling ratio of only approximately 70% in 30 minutes is not acceptable for the purposes of this study.

### Porosity Measurement

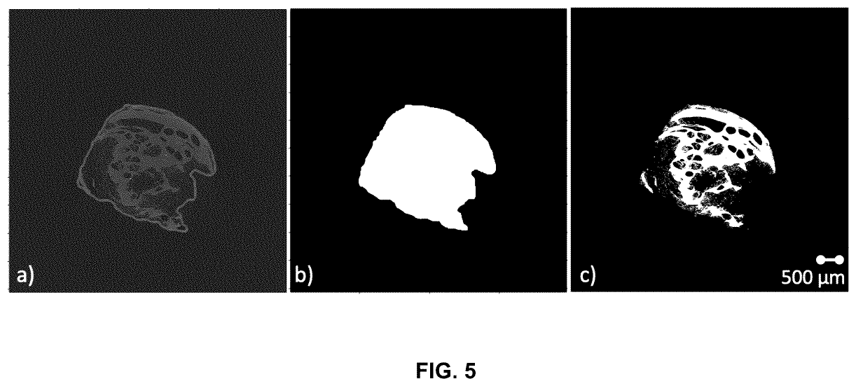

To try and identify why the hydrogels had such limited swelling, the porosity of the Sample 6 hydrogel was analyzed using the micro-CT method, as shown in FIG. 5. There are a few large pores that can be seen on the inside of the hydrogel, likely due to the bubbles generated from the addition of sodium bicarbonate during the formation process. Examining the entire set of micro-CT scan images made it clear that the majority of these pores were closed pores: pores that do not connect to other pores or to the outside of the hydrogel. While these closed pores contribute to the overall porosity of the hydrogel, they are largely inaccessible to the swelling media and offer minimal support in decreasing the equilibrium swelling time. Aside from the large pores, the remainder of the hydrogel appears to be very dense, showing little to no porosity in the actual gel structure. The total porosity of the sample was calculated to be approximately 49%, including the closed pores. This lack of porosity was probably what caused the very slow swelling speed of the hydrogels. This led to the conclusion that in order to be effective for acid relocation, a hydrogel with a much higher porosity must be used, shifting the research from gas-blown hydrogels to freeze-dried hydrogels.

### Example 2. Freeze-Drying Method

### Materials

Chitosan (medium molecular weight, 75-85% deacetylated), zirconium (IV) oxide (5 micron diameter), glutaraldehyde solution (25% in water), sodium chloride, and pepsin (from porcine gastric mucosa) were purchased from Sigma Aldrich. Hydrochloric acid (2.0N), glacial acetic acid, and the Cytotoxicity Assay Kit were purchased from VWR. All aqueous solutions were prepared with RO purified water, and all other chemicals used were of analytical grade.

### First Generation

A first generation of hydrogel composites of the type illustrated in FIG. 1 was synthesized using a freeze-drying method. The polymer 14 comprises chitosan. The high-density material 18 comprises zirconia (zirconium dioxide). The cross-linking agent 22 comprises glutaraldehyde.

Two solutions of chitosan with different concentrations were tested and compared. Both 1% w/v and 2% w/v solutions of chitosan were prepared by dissolving chitosan in 1% v/v acetic acid. These chitosan solutions were combined with 1% aqueous glutaraldehyde and various quantities of zirconia (zirconium dioxide) before being mixed using a vortex mixer. After the mixtures were well combined, they were left to sit for 30 minutes to crosslink before being placed into a −80-degree Celsius freezer. Once the hydrogels were adequately frozen, they were placed into a freeze dryer (Labconco™ FreeZone 1 L Benchtop Freeze Dry System) and lyophilized for 12 to 24 hours depending on the sample size.

The different hydrogel formulations used for the initial test are shown in Table 3. The mass of zirconia added to the samples was incremented by units of 5% in relation to the total volume of the sample. This was done to determine the mass of zirconia required to raise the density of the hydrogels above the density of gastric fluid. Based on preliminary trials, it was found that testing 1% w/v chitosan samples with zirconia percentages higher than 20% was excessive and that testing 2% w/v chitosan samples with zirconia percentages less than 25% was insufficient, which is why the samples were allocated accordingly. A standardized volume of 0.12 ml of 1% glutaraldehyde solution was added to each 1 ml sample to act as the crosslinking agent (12% w/w glutaraldehyde to chitosan for 1% chitosan samples, and 6% w/w glutaraldehyde to chitosan for 2% chitosan samples). All experiments were performed in triplicate, and the data presented in the results section are the average values of each of the three trials.

TABLE 3
Freeze-dried hydrogel formulations: chitosan
percentage versus amount of zirconia

| | 10% | 15% | 20% | 25% | 30% | 35% |
|---|---|---|---|---|---|---|
| | Zirconia | Zirconia | Zirconia | Zirconia | Zirconia | Zirconia |
| | (w/v) | (w/v) | (w/v) | (w/v) | (w/v) | (w/v) |
| 1% | Sam- | Sam- | Sam- | — | — | — |
| Chitosan | ple 1 | ple 2 | ple 3 | | | |
| 2% | — | — | — | Sam- | Sam- | Sam- |
| Chitosan | | | | ple 4 | ple 5 | ple 6 |

### Second Generation

A second generation of hydrogel composites of the type illustrated in FIG. 1 was synthesized using a freeze-drying method. The second generation of freeze-dried hydrogels was developed based on the results from the first generation of freeze-dried hydrogels. The methodology used to prepare the hydrogels was kept identical as the methodology used to prepare the first-generation hydrogels; however, the concentrations of glutaraldehyde and zirconia were modified to attempt and improve the biocompatibility and swelling abilities of the hydrogels. An initial test was performed using reduced quantities of 1% aqueous glutaraldehyde solution, comparing 2% w/w, 1% w/w, 0.5% w/w, and 0.25% w/w concentrations of glutaraldehyde to chitosan. After the ideal percentage of glutaraldehyde was determined, a final test was performed to identify the amount of zirconia required to raise the density of the improved swollen hydrogel above the density of gastric fluid.

### Results—First Generation

### Hydrogel Synthesis

The first-generation freeze-dried hydrogels presented with varying degrees of gelation prior to the lyophilization process, depending on the percentage of chitosan. The 1% chitosan samples, both with and without zirconia, remained in the liquid phase after the addition of the glutaraldehyde, whereas the 2% chitosan samples congealed into a solid mass after approximately 10 minutes of continual stirring. To determine if the gelation was time based, three 1% chitosan samples were kept at room temperature for 24 hours after the stirring process and reassessed. Even after the 24-hour crosslinking period, the 1% chitosan samples maintained a liquid phase and did not present with any visible change in viscosity. Based on this and the gelation time of the 2% percent samples, the 30-minute gelation time was deemed satisfactory for further tests. The combination of these gelation responses implied that the amount of glutaraldehyde present in the formulations was more than what was required for the adequate crosslinking of the chitosan within the hydrogels. This also suggested that the 1% chitosan samples did not have enough polymer to completely gel during the crosslinking process.

After lyophilization, structural differences could be identified between the various samples. Unlike the gas-blown hydrogels, all of the freeze-dried samples maintained their original volumes during the water removal process and had very spongy appearances. Depending on the quantity of chitosan and zirconia in the hydrogel, the samples presented with varying degrees of brittleness and rigidity. The 1% samples without zirconia portrayed the lowest rigidity and the 2% samples with 35% zirconia portrayed the highest rigidity. The addition of zirconia into the polymer network increased the structural integrity of the 1% samples, preventing them from being easily ripped apart while in hydrochloric acid; however, in the higher concentration 2% samples, the large amounts of zirconia resulted in extremely brittle and hard hydrogels.

### Swelling Studies

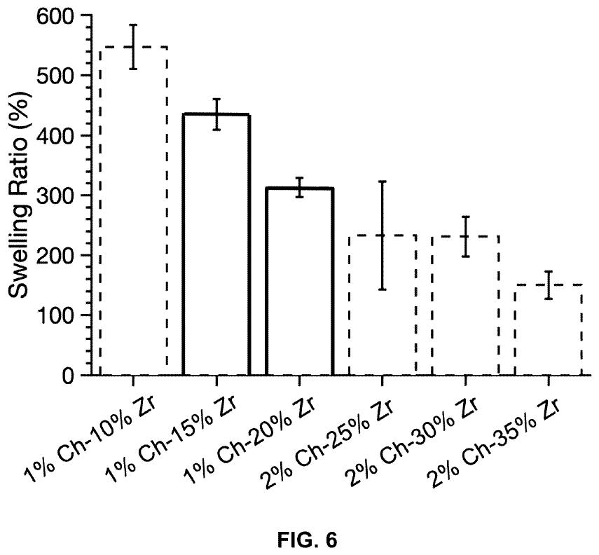

The results of the initial swelling test in pH 1.57 hydrochloric acid (FIG. 6) outlined two potential candidates: Sample 2 and Sample 3, as these samples were the only ones that possessed a density higher than gastric fluid. A clear relationship can be observed between the amount of zirconia added to the hydrogel and the swelling ability, but as the swelling ratio is calculated based on weight, this is to be expected. The difference in hydrogel mass before and after swelling was compared with ANOVA and was found to be insignificant within each chitosan percentage group (a=0.05). This suggests that the addition of zirconia has no impact on the swelling capabilities of the hydrogels. It can also be seen that an increased percentage of chitosan has a negative impact on the swollen density of the hydrogels, as none of the 2% samples were capable of sinking in gastric fluid even with an additional amount of zirconia up to 35%. This is likely a result of the amplified crosslinking density of the polymer structure that occurs with the use of a greater concentration of chitosan. Due to the strengthened attachments that develop between polymer chains, the pores generated during the lyophilization process of a highly crosslinked hydrogel will be smaller than the pores generated in a hydrogel with a lower crosslinking density. This makes it more difficult for media to penetrate the hydrogel structure, resulting in reduced swelling abilities. Additionally, the chitosan consumes more of the sample volume in the 2% chitosan hydrogels, decreasing the overall density of the hydrogel due to the low density of chitosan (0.2-0.38 g/ml). Based on the findings from this experiment, Sample 2 appeared to have optimal polymer and zirconia concentrations for maximized gastric acid relocation and was selected for further analysis. These results also indicated that an additional mass of 15% w/v was required to cause the necessary density increase in the hydrogels and that the maximum swelling ratio that could be attained was approximately 435%.

The swelling response of the Sample 2 hydrogel was also analyzed in pH 1.2 hydrochloric acid, pH 1.2 simulated gastric fluid, and pH 7.0 water. The average swelling ratios of the samples in pH 1.2 hydrochloric acid and simulated gastric fluid yielded a difference in swelling response of less than 0.2% and ANOVA determined that this difference was insignificant (a=0.05). This suggests that the presence of pepsin and sodium chloride in gastric fluid had a negligible effect on the swelling capabilities of the hydrogel. The overall swelling ratio in pH 1.2 media was calculated to be approximately 437%, only 2% larger than in pH 1.57 media; however, the swelling ratio of the hydrogel in water was reduced by −18%. At low pH values, the amine groups on the chitosan polymer are protonated, resulting in the polymer becoming positively charged. These positive charges within the hydrogel network cause segments of the polymer to repel against one another, encouraging further swelling in the hydrogel. As this effect only occurs at low pH values, the swelling was reduced in pure water.

### Porosity Measurement and Calculation of Density

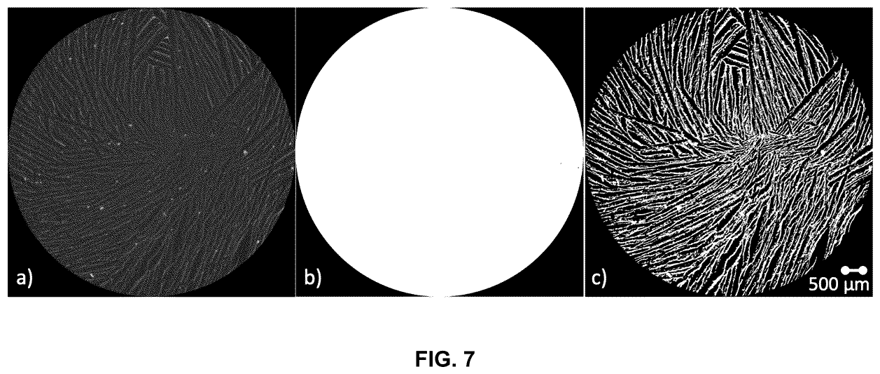

The freeze-dried hydrogels had highly porous structures as depicted in FIG. 7. Scanning through the micro-CT images outlined that the pores within the freeze-dried hydrogels were interconnected and open. This allows for fluid to permeate the hydrogels through capillary wetting instead of diffusion, resulting in extremely fast and significant swelling. This interconnected porous structure was likely the reason for the dramatic increase in swelling ability compared to the gas-blown hydrogels. Large, stacked pores can be observed running down the length of the hydrogel in the axial direction. Although the pores are quite narrow, they appear to vary in length from approximately 500 μm to 4000 μm, allowing for the freeze-dried hydrogels to be classified as superporous hydrogels.

The results from both the hexane-based porosity test and micro-CT-based porosity analysis are shown in Table 4. The results from the hexane-based porosity test indicate that the addition of zirconia into the hydrogel increased its porosity. This is a common finding in hydrogels that have metal oxides incorporated into their structure, and is specifically noted for titanium dioxide. It is hypothesized to occur from the inorganic material disrupting the crosslinking process, resulting in a more porous hydrogel overall, even though there is a decrease in pore width. While the hexane-based porosity values indicated how the zirconia impacts the porosity of the hydrogel, the micro-CT-based values were considered to be more accurate due to the high evaporation rate of hexane. Due to this, the micro-CT-based values were selected for further calculations for the 15% zirconia hydrogel. The densities of the hydrogels with and without zirconia were calculated using the optimal porosity values. As expected, the swollen density of the Sample 2 hydrogel was calculated to be 1.01 g/ml, confirming its ability to sink in gastric fluid.

TABLE 4
Hydrogel porosity and density comparison of
first-generation freeze-dried hydrogels

| | | Apparent | | Swollen |
|---|---|---|---|---|
| | Porosity | Density | Density | Density |
| | (%) | (g/ml) | (g/ml) | (g/ml) |
| Sample 2, | 71.2* | 0.01 | 0.033* | 0.858 |
| without Zirconia | | | | |
| Sample 2, with | 90.5* \| 72.1** | 0.153 | 0.550** | 1.01 |
| Zirconia | | | | |

*Calculated using the hexane-based method
**Calculated using the micro-CT-based method

### Scanning Electron Microscopy

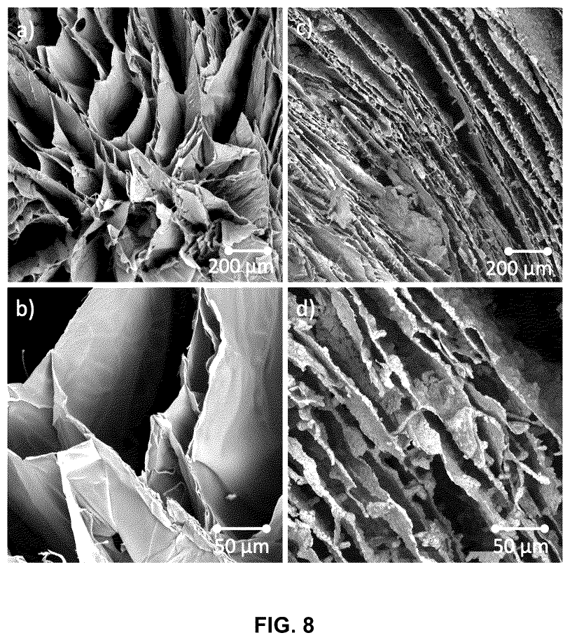

The SEM images (FIG. 8) show how the zirconia impacted the inner structure of the hydrogel. Highly porous structures are clearly visible in both types of hydrogels; however, it was observed that the zirconia altered the pore structure that developed during the lyophilization process. The hydrogels without zirconia expressed a honeycomb or sponge-like pore structure, whereas the hydrogels with zirconia expressed a flatter and more compressed pore structure, which was assembled in thin layers. Sponge-like pores are a common occurrence in chitosan-based freeze-dried hydrogels. Sponge-like pores occur due to the lyophilization process; as a hydrogel sample freezes, ice crystals will nucleate and grow along the lines of thermal gradients, resulting in a specific pore pattern after the ice has been removed from the sample. While the hydrogels with zirconia still appeared to form based on these thermal gradients, the pores were much less wide (a change of approximately 200 μm to 25 μm).

Instead of resting inside the pores of the hydrogel, the zirconia particles appeared to be bonded to the polymer network. This may have occurred because the zirconia was weakly bonded to the chitosan through hydrogen bonds. The observation is consistent with the experimental results because if the zirconia was not adequately connected to the polymer network, it would precipitate out of the hydrogel upon submersion in liquid. The effective incorporation of zirconia into the hydrogel is extremely beneficial because even in this highly porous state, additional chemicals or techniques are not required to help bond the zirconia to the polymer network.

### Fourier Transform Infrared Spectroscopy

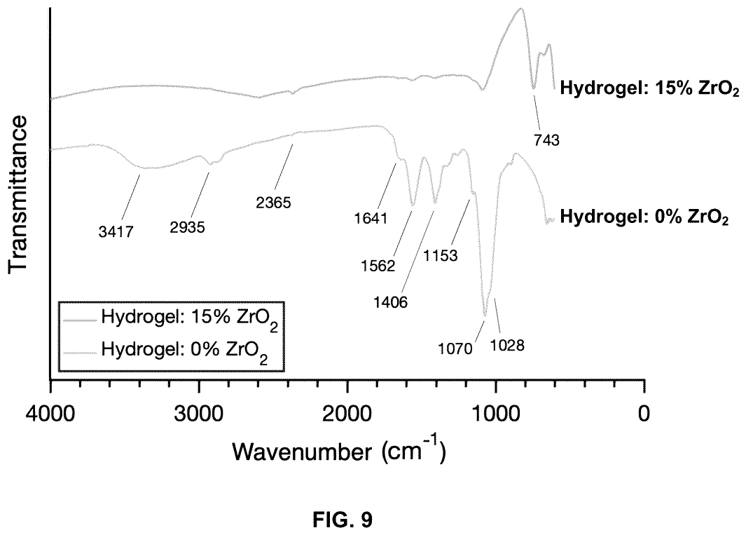

The FTIR results are shown in FIG. 9. In the pure chitosan hydrogel, a broad band could be noted at 3417 cm⁻¹ which correlates to O—H and N—H stretching and small sharper bands were seen at 2935 cm⁻¹ and 2365 cm⁻¹ which are attributed to stretching in the CH2 aliphatic groups. The small shoulders at 1153 cm⁻¹ and 1028 cm⁻¹ as well as the peak at 1070 cm⁻¹ are due to the asymmetric stretching of the C—O—C bridge in the glycosidic bonds. C═N stretching in the imine bonds is responsible for the small shoulder present at 1641 cm⁻¹ and the peak present at 1562 cm⁻¹ is due to stretching in the C═C bonds. Additionally, the peak at 1406 cm⁻¹ is from stretching vibrations in the C—N bonds. These various peaks are commonly noted in glutaraldehyde-crosslinked chitosan hydrogels, suggesting that the general hydrogel preparation methodology used was appropriate and that the hydrogels were chemically crosslinked. The sharp peak at 743 cm⁻¹ that was only present in the hydrogel with zirconia is caused by Zr—O bending vibrations. Although small, many of the chitosan-glutaraldehyde peaks could be recognized in the hydrogel with zirconia. The reasoning that the chitosan-glutaraldehyde peaks had very low amplitudes in the hydrogel with zirconia may be that the specific sample of hydrogel taken for analysis was primarily composed of zirconia. At 15% w/v, the mass of the sample after freeze-drying was more than 90% due to the added zirconia. Since the sample must be broken down into a powder for FTIR analysis, there is a high likelihood that the majority of this powder was zirconia, resulting in very little of the chitosan-glutaraldehyde polymer actually being analyzed.

### Cell Cytotoxicity Study

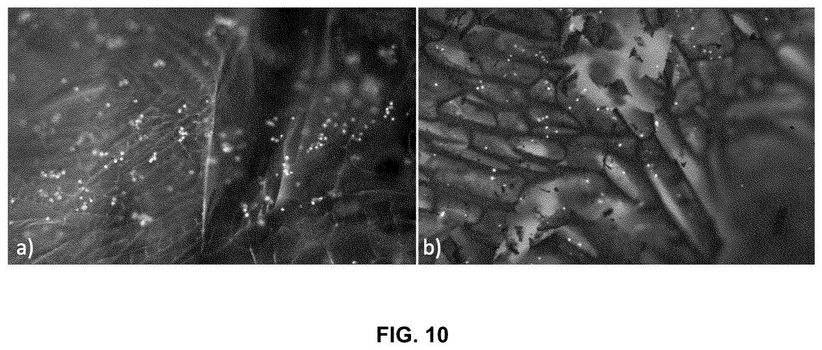

The cytocompatibility of the hydrogels was assessed under fluorescence microscopy, where living cells were viewed as the colour green and dead cells were viewed as the colour orange or red. The hydrogels were first observed under an optical microscope, where the cells were evaluated for their elongation and growth. This evaluation took place one day after the cells had been seeded onto the surfaces of the hydrogels. It was found that while the cells in the control group had elongated, the cells that were seeded onto the hydrogels had not elongated or multiplied. To determine if the hydrogels merely hindered the multiplication process of the cells causing them to grow slower than normal, the samples were stained with fluorescent dye and the cells were observed under a fluorescent microscope (FIG. 10). It can be seen that the cells did not elongate in either sample, noted by their small circular shapes. In addition, the colour of the cells was light orange in both samples, indicating that the cells had died. As both samples demonstrated similar cytotoxicity, it can be inferred that the addition of the zirconia was not the sole factor that resulted in the death of the cells.

Based on other studies conducted using glutaraldehyde-crosslinked chitosan, it was concluded that the likely cause of the failed cell toxicity study was excess glutaraldehyde. The hydrogels presented in this subchapter were made with a 1% solution of glutaraldehyde, however, the mass of the glutaraldehyde was calculated to be 12% of the mass of the chitosan when using a 1% chitosan solution.

### Results—Second Generation

### Hydrogel Synthesis

Prior to lyophilization, the hydrogel samples appeared to show similar degrees of gelation after the crosslinking process. Due to the limited amount of glutaraldehyde used and low concentration of chitosan, solid hydrogels were never formed and the samples remained completely liquid until placement in the freezer. After lyophilization, the hydrogels were extracted from the falcon tubes and physical differences could be observed between the different sample sets. While the hydrogels all expressed the same general appearance, some of the zirconia had pooled at the bottom of the falcon tubes and separated from the hydrogels. Hydrogels that were made with higher percentages of glutaraldehyde were able to support larger amounts of zirconia within the hydrogel structure, resulting in an initial difference in weight between the hydrogel samples.

There was a noticeable difference in colour that could be observed between the first-generation and second-generation hydrogels. The crosslinked chitosan in the first-generation of hydrogels expressed a pink/orange tone, whereas the second-generation of hydrogels had a very pale-yellow colour. It was originally thought that this pink hue developed from a reaction within the crosslinking process, causing the natural colour of chitin to be expressed. As the percentage of chitosan used in both versions of the hydrogel was identical, this colour change must have been due to the difference in glutaraldehyde concentration, suggesting that excess glutaraldehyde created pink and orange tones within the chitosan structure.

### Swelling Studies

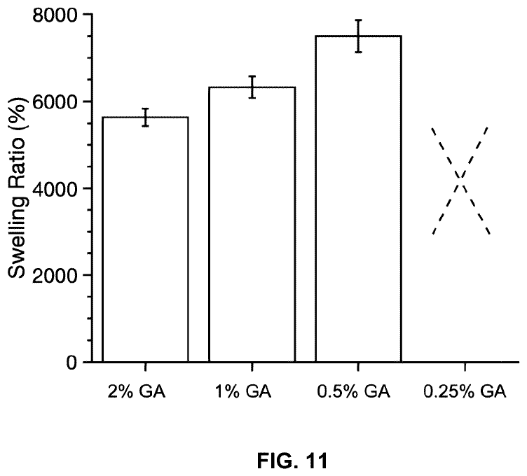

The results from the initial 30-minute swelling experiment are listed in Table 5. Each of the samples possessed a density higher than that of gastric fluid, even after the excess zirconia had been removed from the hydrogel samples. As the swelling ratio is largely dependent on the initial weight of the sample, the fact that the swelling ability of the hydrogels increased as the amount of zirconia remaining in the hydrogels decreased was to be expected; however, a relationship between the swelling ability and the degree of crosslinking was also present. It can be noted that regardless of the initial weight of the hydrogel, the swollen mass of the hydrogels increased as the amount of the glutaraldehyde decreased, suggesting that samples with less glutaraldehyde could swell more. This may suggest that hydrogels with a lower crosslinking density have greater swelling capabilities. As it was found that zirconia does not impact the swelling abilities of the hydrogels, a new value termed the expectant swelling ratio was calculated for each of the samples to better visualize the relationship between swelling ability and the volume of crosslinker (FIG. 11). The expectant swelling ratio was defined as the swelling ratio that the hydrogel would have achieved if zirconia was not introduced into the hydrogel sample. The results from FIG. 11 outline the inverse relationship between the amount of glutaraldehyde and the swelling ratio of the hydrogels.

TABLE 5
Swelling ratios of second-generation freeze-dried hydrogel
samples in pH 1.57 hydrochloric acid: glutaraldehyde study

| | | Volume of | Mass of | | | Swell- |
|---|---|---|---|---|---|---|
| | Volume of | Glutaral- | Zirconia | Initial | Swollen | ing |
| | Chitosan | dehyde | Added | Mass | Mass | Ratio |
| | (ml) | (ml) | (g) | (g) | (g) | (%) |
| Sample 1 | 1 | 0.02 | 0.15 | 0.0857 | 0.83 | 868 |
| Sample 2 | 1 | 0.01 | 0.15 | 0.0885 | 0.9238 | 943 |
| Sample 3 | 1 | 0.005 | 0.15 | 0.0609 | 1.0508 | 1625 |
| Sample 4 | 1 | 0.0025 | 0.15 | 0.0533 | —* | —** |

* & ** The swollen mass and swelling ratio of Sample 4 is left blank because the sample disintegrated upon submersion in the hydrochloric acid solution.

The degree of crosslinking in Samples 1, 2, and 3 was found to be sufficient as the hydrogels maintained a solid structure within the hydrochloric acid, but this was not the case for Sample 4, which immediately disintegrated upon contact with the solution. Based on these results, it was concluded that the ideal volume of glutaraldehyde for the 1 ml samples was 0.005 ml, or a 0.5% w/w (glutaraldehyde to chitosan) mixture. It was also concluded that the amount of zirconia in the hydrogel network could likely be further decreased as a means of increasing the swelling ratio of the hydrogels. This inspired a second set of experiments to determine the ideal amount of zirconia, the results of which are shown in Table 6.

TABLE 6
Swelling ratios of second-generation freeze-dried hydrogel
samples in pH 1.57 hydrochloric acid: zirconia study

| | | Volume of | Mass of | | | Swell- |
|---|---|---|---|---|---|---|
| | Volume of | Glutaral- | Zirconia | Initial | Swollen | ing |
| | Chitosan | dehyde | Added | Mass | Mass | Ratio |
| | (ml) | (ml) | (g) | (g) | (g) | (%) |
| Sample 5 | 1 | 0.005 | 0.055 | 0.0605 | 1.07 | 1673 |
| Sample 6 | 1 | 0.005 | 0.05 | 0.05198 | 1.08 | 1987 |
| Sample 7 | 1 | 0.005 | 0.045 | 0.05065 | 1.12 | 2107 |

The results of this set of experiments show that an additional mass of 0.045 grams of zirconia (4.5% w/v) is the ideal amount to add to the hydrogels for maximum swelling efficiency. Efforts made to use less than 0.045 grams resulted in hydrogels with a density lower than gastric fluid and were deemed unsuccessful. Thus, the hydrogel in sample set 7 was determined to have the optimized hydrogel formula and was selected for further study.

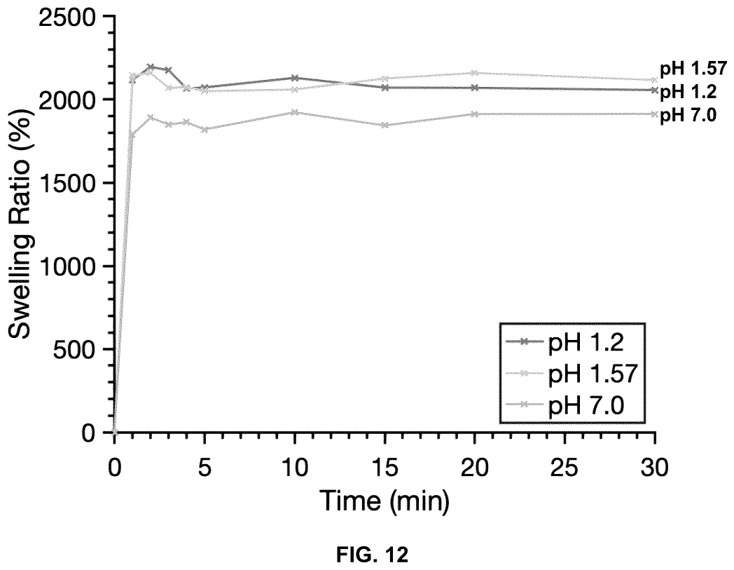

Three additional swelling studies were performed on the Sample 7 hydrogel: determination of equilibrium swelling time, pH sensitivity, and response to compression. The equilibrium swelling time and pH sensitivity tests were combined into one experiment where the swelling of the hydrogel over time was compared for three different forms of media, the results of which are shown in FIG. 12. This experiment indicated that the hydrogel samples were fully swollen after less than a minute in solution. Based on this observation, as well as the change in appearance of the hydrogels during the swelling process, it is likely that the hydrogels were completely swollen by the time the sinking process was complete. After the first minute the hydrogel appears to fluctuate mildly in weight; however, this may be due to the continual interference from removing the hydrogel from the solution to take accurate measurements. A difference in swelling ability can be noticed when comparing the pH 7.0 and pH 1.2 and 1.57 solutions. Initially, there is a 328% difference in swelling ratio from the low pH solutions to the high pH solution, representing the same trend that was depicted in the first-generation hydrogels. The results of the trials in simulated gastric fluid were excluded from FIG. 12 because the pepsin caused the hydrogel to slowly disintegrate due to its chitosanolytic nature. After approximately one minute in the simulated gastric fluid, the pepsin began to dissolve the hydrogel, causing small segments of zirconia to separate from the hydrogel sample. While this indicated that the hydrogel was performing as desired and could be broken down in the stomach prior to moving through the rest of the gastrointestinal tract, it made the data from re-weighing the hydrogel sample unreliable. For the purposes of this study, it was assumed that the swelling response of the hydrogel in simulated gastric fluid was similar to that of the hydrogel in pH 1.2 hydrochloric acid based on the studies done with the first-generation hydrogels and the identical pH values.

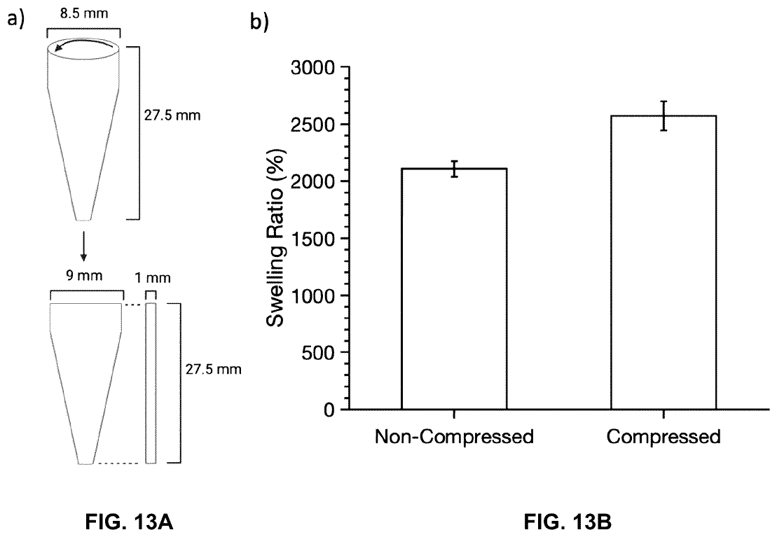

To assess the hydrogel's response to compression, hydrogel samples were flattened to approximately 15% of their original volume (as shown in FIG. 13A) and assessed for swelling ability in pH 1.57 hydrochloric acid (FIG. 13B). It was originally hypothesized that removing the free air from the porous network would impede the swelling abilities of the hydrogels; however, experimental results demonstrated that their swelling abilities improved as a result of the compression. The compressed hydrogels were able to swell about 400% more than the uncompressed hydrogels in the same time span. Based on similar findings from other researchers, it is thought that this increase in swelling ability occurs due to a change in the porous structure of the hydrogels that happens during the compression process. Additionally, the zirconia may be preventing the capillary channels within the hydrogel from completely sealing shut as the hydrogel is flattened, similar to how disintegrants support compressed superporous hydrogels used for drug delivery. This additional support is likely the reasoning as to why the swelling times of the hydrogels were not increased after being compressed. The improved swelling ability of the compressed hydrogels is extremely beneficial when considering placing the hydrogels inside of gel capsules for easier transport to the stomach.

### Porosity Measurement and Calculation of Density

The hydrogels both with and without zirconia were highly porous, showing average porosities of 91% and 88%, respectively, using the hexane-based method and 87% using the micro-CT-based method (Table 7). The different porosity values were compared using ANOVA and the differences between the means were not found to be significant (a=0.05), suggesting that the introduction of zirconia into the hydrogel network at a 4.5% w/v concentration did not greatly impact the porosity of the hydrogel. The estimated swollen density for the hydrogel samples without zirconia implied that the hydrogels did not need the addition of zirconia to sink in gastric fluid; however, experimental results proved that this was not the case. This suggests that the estimated values for the swollen hydrogel densities are likely incorrect by approximately 4%. The minor discrepancies between the predicted and observed swelling densities can probably be attributed to the small initial masses of the hydrogel samples, causing minor variations in weight to be amplified in the results.

TABLE 7
Hydrogel porosity and density comparison of
second-generation freeze-dried hydrogels

| | | Apparent | | Swollen |
|---|---|---|---|---|
| | Porosity | Density | Density | Density |
| | (%) | (g/ml) | (g/ml) | (g/ml) |
| Sample 7, | 88* | 0.0132 | 0.11* | 1.04 |
| without Zirconia | | | | |
| Sample 7, with | 91* \| 87** | 0.0507 | 0.39** | 1.09 |
| Zirconia | | | | |

*Calculated using the hexane-based method
**Calculated using the micro-CT-based method

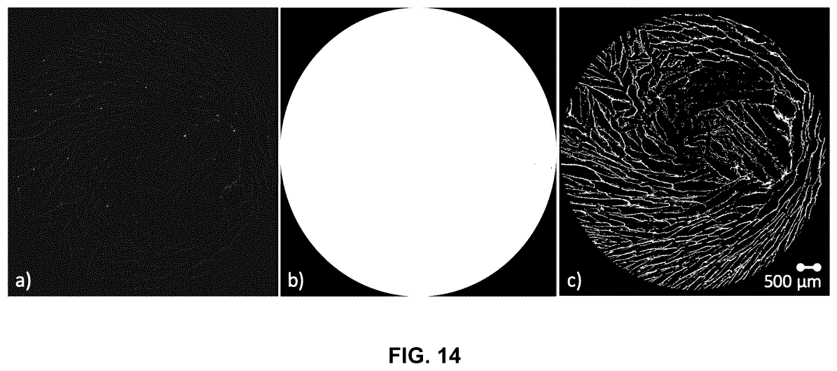

An image from the micro-CT scan is shown in FIG. 14. It can be seen that the pores developed in the second-generation hydrogels were less linear and more open than in the first-generation of hydrogels, possessing pores ranging from 100-300 μm in size. This change in pore shape was likely primarily due to the decrease in zirconia from the first-generation hydrogels to the second-generation hydrogels, as the cross-section depicted here seems to resemble the SEM image of the first-generation hydrogel without zirconia. The increase in pore width likely contributed to the increased swelling abilities of the second-generation hydrogels.

### Scanning Electron Microscopy

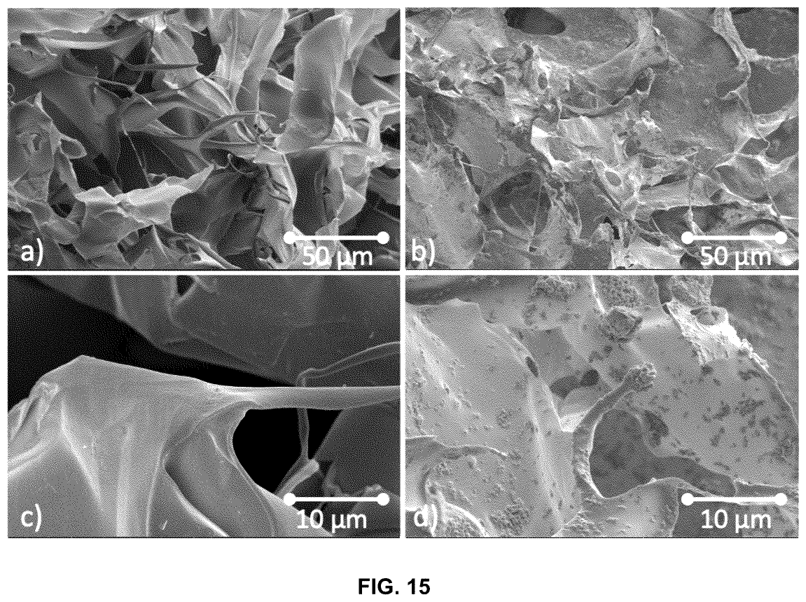

The inner structures of the hydrogels are shown in FIG. 15. Both types of hydrogels are shown to have highly interconnected porous structures made with incredibly thin polymer layers. Unlike the first generation of hydrogels, the addition of zirconia did not seem to change the type of pores that developed in the hydrogel, likely due to the dramatic decrease in zirconia concentration. Although the hydrogels still possessed an incredibly spongy structure, the hydrogel walls appeared to be much thinner and flakier in the second generation of hydrogels. As the only difference between the first- and second-generation hydrogels without zirconia was the concentration of glutaraldehyde, this difference in pore structure demonstrated how the degree of crosslinking affects the overall structure of the hydrogel.

### Fourier Transform Infrared Spectroscopy

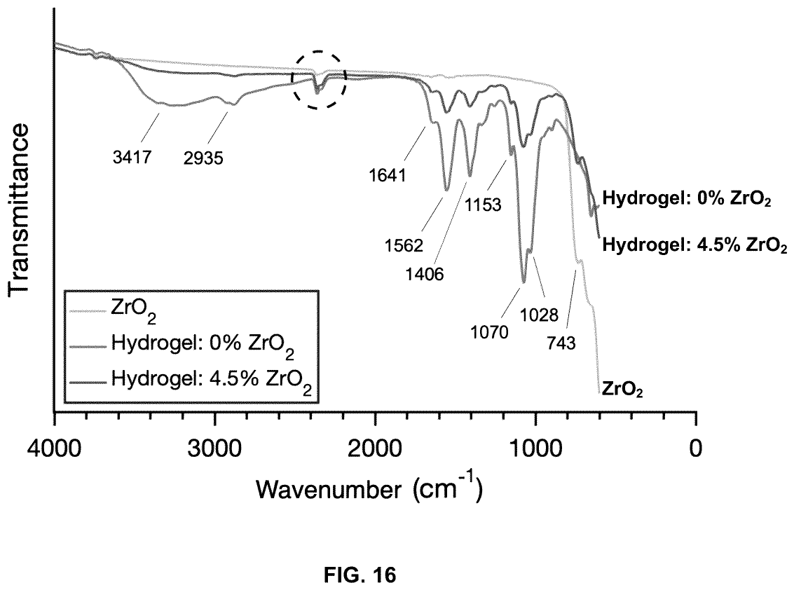

FIG. 16 shows the FTIR results of the second-generation hydrogels and the zirconia powder. The same peaks that were recognized in the original FTIR results of the first-generation hydrogels are all present, but the chitosan-glutaraldehyde peaks are more pronounced in the second-generation zirconia filled hydrogel compared to the first. Additionally, the same peak at 743 cm⁻¹ due to the Zr—O bending vibrations is visible in the 4.5% zirconia hydrogel and confirmed by the analysis of pure ZrO₂. Minor noise in the FTIR spectra occurred at approximately 2300 cm⁻¹, which is likely due to the asymmetric stretching mode of CO₂.

### X-Ray Diffraction

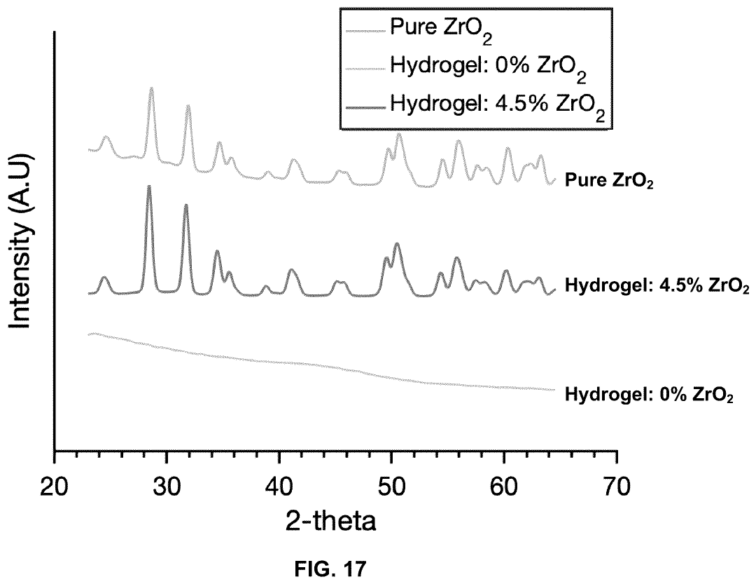

FIG. 17 shows the XRD patterns of pure zirconia, the chitosan hydrogel without zirconia, and the chitosan hydrogel with zirconia. Zirconia has a crystal structure, represented by many sharp peaks over the 2-theta range. These peaks in the zirconia spectrum can be identified at 24.2°, 28.7°, 31.5°, 34.8°, 38.5°, 40.8°, 44.4°, 49.5°, 54.0°, 56.0°, and 60.5°, corresponding to [011], [−111], [111], [200], [120], [−112], [211], [022], [220], [310], [131], and [120] planes, respectively, which are indicative of monoclinic zirconium dioxide. The XRD spectrum of the chitosan hydrogel made without zirconia does not show any peaks due to its amorphous structure and follows the standard general shape for glutaraldehyde-crosslinked chitosan. The peaks associated with the crystal structure of the zirconia can be observed in the XRD spectra of the hydrogel with 4.5% zirconia. As the peaks did not appear to differ in any way, it implied that the crystalline structure of the zirconia was maintained during the hydrogel preparation process.

### Cell Cytotoxicity Study

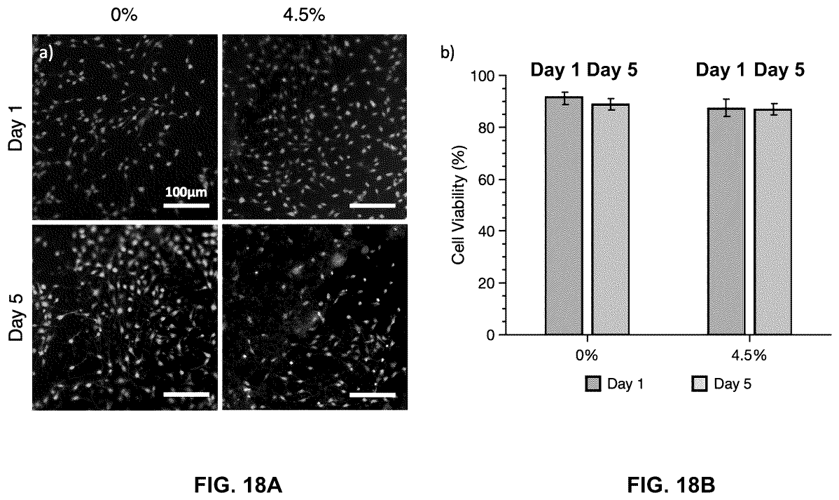

The cytocompatibility of the hydrogels was assessed under fluorescence microscopy where living cells were visible as the colour green and dead cells were visible as the colour orange/red. Images of the stained hydrogels at days 1 and 5 are present in FIG. 18A. The cells in both hydrogels with and without zirconia experienced elongation and growth over the 5-day period. The percentage of cell viability of the 3T3 cells was determined for both samples by image analysis and was calculated to be upwards of 80% (FIG. 18B). The viability of the 3T3 cells in the control group did not significantly differ from the viability of the cells within the hydrogel samples, indicating that both hydrogel varieties are cytologically compatible. Additionally, there was no significant difference in cell viability between hydrogels containing zirconia and hydrogels not containing zirconia, confirming that zirconia was not impacting the cytotoxicity of the samples. These results suggested that the hydrogel has strong potential for in-vivo biocompatibility and may be considered for further study.

### Characterization Techniques

The efficacy of the hydrogels was determined based on four main factors: maximum swelling ability, equilibrium swelling time, swollen density, and biocompatibility. Based on these factors, the studies done on the swelling abilities of the hydrogels were deemed the most important and were used to identify potential candidates for further study. After a sample was selected to move forward with the experimentation process, it was examined through various techniques such as micro-computed tomography imaging, scanning electron microscopy, Fourier transform infrared spectroscopy, x-ray diffraction analysis, and cell cytotoxicity live/dead assays. Formulations that showed limited promise were rejected after only a few characterization techniques, whereas formulations that showed increasing promise were fully studied.

### Swelling Studies

### Initial Feasibility Assessment

The initial feasibility test that was performed on all samples was a swelling experiment in pH 1.57 hydrochloric acid. This specific media was selected to replicate the environment of the gastric acid pocket. The hydrogel samples were weighed and placed in hydrochloric acid for 30 minutes to allow for the hydrogels to swell. Based on the hydrogel type, the samples were periodically extracted, blotted to remove excess fluid, and re-weighed to determine their swollen mass. Hydrogels that were made using the gas blowing method were extracted between one- and ten-minute intervals, whereas hydrogels made using the freeze-drying method were left undisturbed for the full 30 minutes. The reasoning for this is that some of the hydrogels that were made with the freeze-drying method were fragile and tended to break upon repeated removal from the solution. The swelling ratios of the hydrogel samples were calculated according to the formulae as shown below:

$$Swelling Ratio ( \% ) = \frac{{M}_{s} - {M}_{d}}{{M}_{d}} * 100$$

where Mₛ represents the swollen mass of the hydrogel and M_{d} represents the initial dry mass of the hydrogel.

### Additional Swelling Analysis

If a particular formulation of hydrogel exhibited a high degree of swelling in the initial feasibility experiment, additional swelling tests were performed on that sample set. These additional swelling tests included assessing the response to pH variance, the swelling ability in simulated gastric fluid, the swelling rate over time (for freeze-dried formulations), and the response to compression. Three forms of media were used to assess the pH sensitivity of the hydrogels: hydrochloric acid with a pH value of 1.57, hydrochloric acid with a pH value of 1.2, and water with a pH value of 7.0. Hydrochloric acid with a pH of 1.2 was selected to compare with simulated gastric fluid (pH 1.2). This was done to determine the effects that sodium chloride and pepsin have on the swelling ability of the hydrogels. Compressing the hydrogels before analyzing their swelling ability was done to simulate the effects of being compacted inside a gel capsule for easier transfer to the stomach. Hydrogel samples were flattened between two Petri dishes prior to submersion in hydrochloric acid to determine how the swelling abilities of the hydrogel were altered due to the reduced initial volume.

### Porosity Measurement and Calculation of Density

The porosities of the hydrogels were measured in two ways: 1) the solvent displacement method and 2) applying image processing to micro-computed tomography scans. For the solvent displacement method, dried hydrogel samples were weighed and placed in hexane for 30 minutes to allow for the hexane to permeate through the porous structure. After 30 minutes, the hydrogels were weighed again, and their porosities were calculated as shown in the formulae below:

$$P = \frac{{M}_{h} - {M}_{d}}{{ρ}_{h} * {V}_{i}} * 100$$

where Mₕ represents the mass of the hydrogel after being immersed in hexane, ρₕ is the density of hexane, and Vᵢ is the volume of the hydrogel. Using this method, the porosities of hydrogels that contained zirconia were compared against the porosities of hydrogels that did not have zirconia but were otherwise identical. This was done to determine the extent to which the zirconia impacts the porous nature of the hydrogels. Due to the difficulty of working with hexane because of its high evaporation rate, these values were compared against those calculated using the micro-computed tomography image analysis method.

Micro-computed tomography, also referred to as micro-CT, is similar to the type of tomography that is frequently used in hospitals for CAT scans. It is a non-destructive technique that is used to observe the internal structure of an object of interest and has been typically used in the fields of tissue engineering, medicine, and food chemistry, amongst others. Micro-CT uses a beam of x-rays that is repeatedly transmitted through a rotating sample to acquire a series of 2D x-ray projections. These projections can be reconstructed using a filtered back-projection algorithm to create cross-sectional images of the sample. Materials that have a high density will be able to block or absorb most of the x-rays, resulting in a white colouration on the x-ray image, whereas materials that have a lower density will not, causing them to appear grey or black.

The Zeiss microXCT™ 400 scanner was used to obtain cross-sectional images of the samples. Three of the images from each sample set were selected and analyzed in MATLAB where an intensity threshold was placed on the images to separate hydrogel material from free space. Using the binary images gained from the thresholding effect, the number of white pixels, black pixels, and total pixels in the hydrogel area were determined. The porosity was calculated using the formulae:

$$P = \frac{{N}_{B}}{{N}_{r}} * 100$$

where N_{B} is the number of black pixels in the hydrogel area and N_{T} is the total number of pixels in the hydrogel area. Freeze-dried hydrogel samples made without zirconia did not provide enough x-ray opacity to be visible on the micro-CT scans, as a result, only freeze-dried hydrogels with zirconia were analyzed using this method.

Using the determined porosity values, the densities of the hydrogels could be calculated. Three different densities were calculated for the hydrogels: apparent density, actual density, and swollen density, calculated using the following three formula respectively:

$${d}_{α} = \frac{{M}_{d}}{{V}_{i}}$$

$$d = \frac{{M}_{d}}{{V}_{h}} = \frac{{M}_{d}}{( 1 - P / 100 ) * {V}_{i}}$$

$${d}_{s} = \frac{{M}_{s}}{{V}_{i}}$$

where Vₕ, represents the volume of the hydrogel, excluding the free space within the hydrogel structure.

### Scanning Electron Microscopy

The morphologies of the hydrogel samples were studied using a scanning electron microscope (SEM). SEMs are a type of microscope that utilize a beam of electrons instead of photons to form an image, resulting in a much higher resolution than traditional optical microscopes. Electrons are produced at the top of the microscope using an electron gun and are sent down the microscope through a series of apertures and lenses to focus the electrons into a beam. This beam of electrons makes contact with the sample, and as it does, secondary electrons, backscattered electrons, and characteristic x-rays are produced, which can be detected to create images. Samples both with and without zirconia were analyzed to identify any differences in hydrogel structure caused by the zirconia. The cylindrical samples were cut in half to expose the inner structure of the cross-linked polymer and were coated with a thin layer of gold or iridium (Leica EM ACE600™). The first-generation samples were observed using backscattered electrons at a voltage of 20 kV (MIRA3 TESCAN™) and the second-generation samples were observed using secondary electrons at a voltage of 5 kV (FEI Helios™)

### Fourier Transform Infrared Spectroscopy

Fourier transform infrared spectroscopy (FTIR) was used to investigate the compatibility between the chitosan hydrogel and the zirconia particles. FTIR is used to study how infrared light interacts with matter, providing insight into the different molecules present within a sample as well as their respective concentrations. In FTIR, infrared radiation is applied to a sample, which is selectively absorbed at different wavelengths depending on the molecules present within the material. This absorption data can be recorded and converted to an infrared spectrum where different absorption peaks can be observed, corresponding to the molecular fingerprint of the material. The absorption spectra of hydrogels containing zirconia, hydrogels without zirconia, and pure zirconia were compared using a Nicolet™ iS20 FTIR Spectrometer.

### X-Ray Diffraction Analysis

X-ray diffraction analysis (XRD) is a common technique used to identify the crystallographic structure of a material. When an x-ray contacts a solid crystalline object, it is reflected in a particular direction based on the structural orientation of the atoms within the object. The direction and number of these x-rays can be detected, giving light to the different crystal structures within the material. One of the more powerful uses of XRD is the detection of crystalline phases within an amorphous sample, as amorphous materials, such as chitosan, do not produce diffraction patterns with identifiable peaks. In this study, XRD was used to identify the crystal structure of zirconia and determine if incorporating it into the hydrogel resulted in any changes in the XRD pattern. The freeze-dried hydrogel samples were ground down to a fine powder and were analyzed over a 2-theta range of 5-70 degrees using a Rigaku™ Rapid Axis X-ray Diffractometer.

### Cell Toxicity Study

Some of the preliminary tests used to determine if a drug is safe for human interaction are cell cytotoxicity assays. In cell cytotoxicity assays, cells are used to determine the likelihood that a material possesses toxic properties based on the percentage of cells that survive while in contact with the material. A common version of these assays are live/dead viability stains, where one type of dye specifically stains living cells (often resulting in green fluorescence) and another type of dye specifically stains dead cells (resulting in red fluorescence). After being given nutrients and time to grow, the cells can be stained with both dyes and observed under fluorescence, where the number of living and dead cells can be determined. When these values are compared against those of the control group, it can be ascertained if the material causes an increase in cell death, therefore maintaining cytotoxic properties.

In order to assess the biocompatibility of the hydrogels, a cell toxicity study was performed with 3T3 mouse embryonic fibroblast cells. A 24-well tissue culture plate was placed into a biosafety cabinet and was sterilized by UV for 12 hours. After sterilization, 100 μL of pre-crosslinked hydrogel mixture was added to each of the chosen wells, yielding hydrogel layers approximately 1 mm thick. Six wells were filled with hydrogel mixture containing zirconia and six wells were filled with hydrogel mixture that did not have zirconia, resulting in three hydrogels for each test. The culture plate was frozen at −80 degrees Celsius overnight and subsequently placed in a freeze dryer to lyophilize the hydrogel discs. The freeze-dried hydrogel discs were then washed twice with PBS solution and 3T3 cells were seeded onto the surface of the hydrogels at a seeding density of 2×10⁶ cells per well. 300 μL of Dulbecco's Modified Eagle Medium with 10% fetal bovine serum and 1% penicillin-streptomycin was added to each well and was changed every 2 days. The cell-seeded well plate was incubated at 37 degrees Celsius and 5% CO₂ for a total of five days.

After the first day, the cell culture media was replaced, and the viability of the cells was assessed using a Live/Dead cytotoxicity kit (Viability/Cytotoxicity Assay Kit for Animal Live and Dead Cells). A staining solution of 2 μM calcein AM and 4 μM EthD-III was prepared as listed in the cytotoxicity kit protocol and added to half of the cell-seeded wells on the culture plate. The well plate was covered in aluminum foil and the cells were incubated for 30 minutes at room temperature. Following the incubation period, the cell-bound hydrogel surfaces were washed several times with PBS, observed using fluorescence microscopy, and returned to the incubator for the remainder of the five days. On the fifth day, the well plate was once again removed, and the live/dead assay was repeated as previously described. Cell viability data was measured using ImageJ to count the number of live and dead cells.

Throughout the foregoing description and the drawings, in which corresponding and like parts are identified by the same reference characters, specific details have been set forth in order to provide a more thorough understanding to persons skilled in the art. However, well known elements may not have been shown or described in detail or at all to avoid unnecessarily obscuring the disclosure.

As will be apparent to those skilled in the art in the light of the foregoing disclosure, many alterations and modifications are possible in the practice of this invention without departing from the scope thereof. Accordingly, the description and drawings are to be regarded in an illustrative, rather than a restrictive, sense.

1. A hydrogel composite comprising:
a cross-linked polymer; and
a high-density material bonded to the polymer,
wherein a swollen density of the hydrogel composite is greater than a density of gastric fluid.

2. The hydrogel composite according to claim 1, wherein the swollen density of the hydrogel composite is at least 1.05 g/mL.

3. The hydrogel composite according to claim 1, wherein the cross-linked polymer comprises an ionic polymer.

4. The hydrogel composite according to claim 3, wherein the ionic polymer comprises a polar functional group.

5. The hydrogel composite according to claim 4, wherein the polar functional group is one or more of a hydroxyl group (—OH), a carboxyl group (—COOH), and an amino group (—NH₂).

6. The hydrogel composite according to claim 1, wherein the cross-linked polymer comprises a cationic polymer.

7. The hydrogel composite according to claim 6, wherein the cationic polymer is selected from the group consisting of chitosan, poly(ethyleneimine), poly(amino-co-ester)s, poly(amidoamine)s and poly-L-(lysine).

8. The hydrogel composite according to claim 1, wherein the cross-linked polymer is formed by adding a cross-linking agent to a polymer.

9. The hydrogel composite according to claim 8, wherein the cross-linking agent comprises a physical cross-linking agent or a chemical cross-linking agent.

10. The hydrogel composite according to claim 9, wherein the chemical cross-linking agent comprises a dialdehyde compound.

11. The hydrogel composite according to claim 1, wherein the high-density material comprises a density of greater than 2 mg/mL.

12. The hydrogel composite according to claim 1, wherein the high-density material comprises a metal oxide.

13. The hydrogel composite according to claim 1, wherein the hydrogel composite comprises a plurality of interconnected pores.

14. The hydrogel composite according to claim 1, wherein the porosity of the hydrogel composite is at least 70%.

15. The hydrogel composite according to claim 1, wherein the average pore size of pores in the hydrogel composite is in the range of from about 100 μm to 300 μm.

16. The hydrogel composite according to claim 1, wherein the swelling ratio of the hydrogel composite is greater than about 1500%.

17. The hydrogel composite according to claim 8, wherein the concentration of the cross-linking agent is not greater than 1% w/w relative to the polymer.

18. The hydrogel composite according to claim 1, wherein the concentration of the high-density material is not greater than 15% w/v.

19. Use of a hydrogel composite according to claim 1 as a treatment for heartburn.

20. Use of a hydrogel composite according to claim 1 to relocate gastric acid to a bottom of the stomach.
